(19) United States
(12) Patent Application Publication
(10) Pub. No.: US 2021/0137437 A1
(43) Pub. Date: May 13, 2021

(54) CVC LINE BLOOD DRAWING METHOD

(71) Applicant: JASON HOLLIS, CLIO, MI (US)

(72) Inventor: JASON HOLLIS, CLIO, MI (US)

(21) Appl. No.: 17/151,107

(22) Filed: Jan. 15, 2021

Related U.S. Application Data
(62) Division of application No. 16/053,544, filed on Aug. 2, 2018.

(51) Int. Cl. A61B 5/15

(52) U.S. Cl. CPC A61B 5/150992; A61M 25/0097; A61B 5/150267; A61B 5/150328

(57) ABSTRACT

A method of exchanging fluids through a CVC hub includes a base member with a plurality of ports for the acceptance of a syringe. A top member is rotatably coupled to the base member such that the base member and its associated syringes are configured to rotated relative to the top member. An attachment device is included in communication with the top member. The attachment device releasably coupled directly to the CVC hub. The syringes are selectively engaged with an internal seat in the top member and brought into fluid communication with the attachment device and corresponding CVC hub. Fluids may be exchanged or passed through the attachment device and the syringes from the patient's body.

### BACKGROUND

### 1. Field of the Invention

The present application relates to an assembly for the capture of blood from a patient, and more particularly to a disposable and sterile multi syringe assembly for the capture of blood while reducing the ability of bacteria and virus introduction to enter a central venous catheter along with reducing blood waste.

### 2. Description of Related Art

A central venous catheter (CVC) is also known as a central line, central venous line, or central venous access catheter. A CVC is a catheter that is placed into a large vein of the body, typically around the neck (internal jugular vein), chest (subclavian vein or axillary vein), groin (femoral vein), or through veins in the arms (also known as a PICC line). CVC lines are used to administer medication or fluids that are unable to be taken by mouth or would harm a smaller peripheral vein. They are also used to obtain blood tests and measure central venous pressure.

Present methods of drawing blood from a patient through a CVC include multiple steps where each step introduces a chance of contamination into the patient's blood stream. Additionally the method necessitates the unneeded waste or discarding of blood from the patient. This method uses 3 separate syringes.

During this method, a CVC line is attached to a patient. A first syringe with 10 cc of normal saline is located in the CVC hub and used to flush the CVC line. The term “flush” refers to a solution of saline that is used to clear the CVC line of anything that was being infused in the patient. After flushing, a quantity of fluid is drawn back (i.e. 10 cc of waste) into the syringe. The waste refers to a mixture of patient blood and saline solution involved in the flushing done in the prior step. The first syringe is removed and replaced with a second syringe. The second syringe is located in the CVC hub and draws back between 1 cc and 20 cc of fresh undiluted blood. The second syringe is then removed and replaced with a third syringe containing 10 cc of normal saline. This saline is flushed through the CVC. The third syringe is then removed.

Each time a syringe is removed and replaced, opens up an opportunity for contamination. That is 3 possibilities of introducing a bacteria or virus into the patients' blood stream. Each syringe detaches from and makes contact with the CVC directly. This method also wastes 10 cc of the patients' blood every time blood is drawn, which could be as often as every two hours. So, this method allows two negatives: multiple opportunities for introduction of something undesirable into the patients' bloodstream, and it does not promote blood conservation.

Although strides have been made to provide effective and efficient practices and tools in the medical industry, shortcomings remain. It is desired that an assembly be provided that allows for the selective communication of syringes with a CVC without repeated detachment with the CVC itself. It is desired that the assembly conserve blood of the patient and minimize the chance of infection.

### SUMMARY OF THE INVENTION

It is an object of the present application to provide a blood drawing assembly for a CVC line. The assembly is configured to utilize a single attachment device for communication with the CVC line and selectively alternate syringes between the single attachment device. This allows a single communication with the CVC hub despite the use of multiple syringes. In fact, multiple syringes are alternated through with a single attachment device in communication with the CVC hub.

It is a further object of the present application that the assembly permit for the return of flushed “waste” back into the patient. Additionally, at least one syringe is removable from the assembly for the purpose of filling vials used to transfer blood to a lab or other facility.

The assembly will utilize a rotating base configured to rotate relative to a top member. The single attachment device is in communication with the top member while the syringes are in communication with the rotating base. A receiving hole is located on a bottom surface of the top member for engagement of a syringe. The syringes are actuated through the receiving hole by rotating them within the base.

Another object of the present assembly is the ability to secure the rotational movement of the top member and the base. The base includes in one embodiment an indent and the top member includes a protrusion that selectively aligns with the indent. Rotational movement of the base relative to the top member disengages the indent and protrusion and selectively realigns the protrusion with another indent. One or more protrusions and indents may be used.

The method of using the assembly involves the single connection of the CVC hub with the attachment device. Operation of the syringes is used in a predefined order wherein the set of syringes are rotated about an axis and brought in selective communication with the attachment device. Each syringe is rotated to engage with the attachment device and subsequently rotated for disengagement. When all are disengaged, rotation of the syringes is possible. At least one syringe is fully removable from the base. Ultimately the invention may take many embodiments. This assembly overcomes the disadvantages inherent in the prior art.

The more important features of the assembly have thus been outlined in order that the more detailed description that follows may be better understood and to ensure that the present contribution to the art is appreciated. Additional features of the assembly will be described hereinafter and will form the subject matter of the claims that follow.

Many objects of the present assembly will appear from the following description and appended claims, reference being made to the accompanying drawings forming a part of this specification wherein like reference characters designate corresponding parts in the several views.

Before explaining at least one embodiment of the assembly in detail, it is to be understood that the assembly is not limited in its application to the details of construction and the arrangements of the components set forth in the following description or illustrated in the drawings. The assembly is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and assemblies for carrying out the various purposes of the present assembly. It is important, therefore, that the claims be regarded as including such equivalent constructions insofar as they do not depart from the spirit and scope of the present assembly.

### DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a front view of a blood drawing assembly for a CVC line according to an embodiment of the present application.

FIG. 2 is a bottom view of the blood drawing assembly of FIG. 1.

FIG. 3 is a front view of a syringe in the blood drawing assembly of FIG. 1.

FIG. 4 is an enlarged partial section front view of the blood drawing assembly of FIG. 1.

FIG. 5 is a perspective section view of the blood drawing assembly of FIG. 1.

While the assembly and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE PREFERRED EMBODIMENT

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the assembly described herein may be oriented in any desired direction.

The assembly and method in accordance with the present application overcomes one or more of the above-discussed problems commonly associated with common CVC line practices discussed previously. In particular, the assembly captures a plurality of syringes in a rotating device that is configured to selectively provide for their respective engagement with an attachment device that is in communication with a CVC hub. The assembly is able to alternate syringes without detaching from the CVC hub. These and other unique features of the assembly are discussed below and illustrated in the accompanying drawings.

The assembly and method will be understood, both as to its structure and operation, from the accompanying drawings, taken in conjunction with the accompanying description. Several embodiments of the assembly may be presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

The assembly and method of the present application is illustrated in the associated drawings. The assembly includes a top member and a rotating base member. The top member is in communication with an attachment member configured to communicate directly with a CVC hub. The attachment member includes a hose that passes internally to the top member. The base member includes a plurality of ports for acceptance of a plurality of syringes. The syringes are configured to selectively alternate between a recessed position below the top surface of the base member and an extended position wherein the top of the syringes rise above the top surface of the base member. The top member includes a receiving hole on a bottom surface that aligns with any one of the plurality of ports. The base member may be selectively rotated to align any one of the plurality of syringes with the receiving hole. When aligned, the syringe in the associated port may transition to an extended position so as to engage the tube and be in fluid communication with the attachment member. Multiple rotations and positioning of the various syringes may be performed with the assembly. Additional features and functions of the assembly are illustrated and discussed below.

Referring now to the Figures wherein like reference characters identify corresponding or similar elements in form and function throughout the several views. The following Figures describe the assembly of the present application and its associated features. With reference now to the Figures, an embodiment of the modular observation assembly and method of use are herein described. It should be noted that the articles “a”, “an”, and “the”, as used in this specification, include plural referents unless the content clearly dictates otherwise.

Referring now to FIG. 1 in the drawings, a front view of a blood drawing assembly 101 is illustrated. Assembly 101 is configured to provide a single and continuous attachment device in contact with a CVC hub while simultaneously engaging a plurality of syringes in a sequential manner with a portion of the attachment device, so as to allow fluid communication between a patient's vascular system and at least one of the plurality of syringes. In other words, blood may pass through the CVC hub through the attachment device and selectively engage with any number of syringes. The CVC hub is attached to the patient and has a port to accept the attachment device. The term CVC stands for a central venous catheter that is attached to a patient for the long term transfer of fluids into or out of the body. This decreases the chance of infection as only a single communication is made with the CVC hub despite the use of multiple syringes.

Assembly 101 includes a base member 103, a top member 105, a plurality of syringes 107, and an attachment device 109. Attachment device 109 is coupled at a first end with the CVC hub on the patient and at a second end with top member 105. Base member 103 is configured to hold syringes 107 and selectively rotate relative to top member 105 so as to permit the engagement of each syringe 107 with the second end of attachment device 109. When engaged, there exists fluid communication between the vascular system of the patient and the syringe 107 for the exchange of fluids.

Attachment device 109 has a first end 111 and a second end 113. A port 115 is located at first end 111 and is configured to releasably couple to the CVC hub. Port 115 is a lumen attachment piece used to connect a hollow line 117 between the CVC hub and the plurality of syringes 107. Hollow line 117 extends away from port 115 at first end 111 and passes down through top member 105 and ends at second end 113 internally within a portion of top member 105. This is shown clearly in FIG. 4 of the drawings.

Top member 105 and base member 103 are configured to rotate relative to one another along opposing surfaces. Rotation is made about axis 119. Rotation may be made in either clockwise or counter clockwise directions. Members 103 and 105 are two separate bodies that are coupled together in a manner that permits relative rotation between them. Base member 103 is configured to hold a plurality of syringes 107. As seen in FIG. 1, syringes 107a, 107b, 107c are shown. It is understood that more or less syringes may be used with base member 103. Base member 103 may be made from any rigid material. An example of one such would be a clear plastic material to permit visual inspection of syringes 107.

Referring now to FIG. 2 in the drawings, a bottom view of assembly 101 is illustrated. Base member 103 includes a fastener 121. Fastener 121 is shown coaxial with axis 119. Fastener 121 is configured to couple base member 103 to top member 105 (see also FIG. 4). Fastener 121 is configured to allow selective rotation between members 103 and 105.

Referring now also to FIG. 3 in the drawings, a side view of an exemplary syringe 107 is illustrated. Syringe 107 represents any of syringes 107a, 107b, and 107c. Syringe 107 includes a top portion 123 and a bottom portion 125. Top portion 123 is configured to be inserted and captured by base member 103. Top portion 123 is threaded and includes outer protruding threads configured to mate with base member 103. These threads permit for the rotation and relative linear movement of syringe 107. Syringes 107 are held by base member 103 parallel to axis 119, such that any linear movement of syringes 107 are parallel with the axis 119. Bottom portion 125 includes plungers 126 that extend internally within the syringe body.

Referring now also to FIG. 4 in the drawings, a partial side section view of assembly 101 is illustrated. In particular, syringes 107 are not illustrated herein. The interior of top member 105 is particularly shown so as to see second end 113. Hollow line 117 passes internally into top member 105 and engages an internal seat 127. Seat 127 and line 117 are configured to move or rotate with top member 105. As seen in the figure, fastener 121 is shown as passing through base member 103 and into top member 105. A swivel 122 is shown surrounding fastener 121, the swivel 122 residing between top member 105 and base member 103. Swivel 122 is configured to attach to fastener 121 and permits members 103 and 105 to move independently of each other.

Referring now also to FIG. 5 of the drawings, a lower perspective view of assembly 101 is provided wherein top member 105 is separated from base member 103. Additionally, syringes 107a, 107b, and 107c are shown through base member 103. As noted above, syringes 107 include externally protruding threads 128. These threads 128 are configured to engage with corresponding thread grooves 129 in base member 103. Thread grooves 129 are grooves passing into an internal surface of base member 103. Base member 103 includes a plurality of ports 131 that are configured to accept syringes 107. Ports 131 are defined as the area of base member 103 that extend from a top surface 133 to the most distal point in thread grooves 129. It is understood that the diameter of ports 131 and locations for syringes 107 may vary. For example, syringes 107 may pass through a cylindrical channel in base member 103 and subsequently engage port 131. Port 131 may be narrower than the cylindrical channel.

In use, each syringe 107 operates between a retracted position and an extended position. As seen in FIG. 5, each syringe 107 is shown in a retracted position wherein each syringe rests wholly below top surface 133. In other words, top portion 123 is concealed within its respective port 131, such that top portion 123 is below top surface 133. Each syringe is configured to rotate within port 131 whereby such rotation translates the syringe linearly so as to protrude above and beyond top surface 133. In this condition, syringe 107 is in an extended position. It is ideal that minimal rotational movement is required to induce the linear motion needed to transition between the fully extended and fully retracted positions. For example, assembly 101 may be configured such that grooves 129 and threads 128 only need a half turn of syringe 107 to fully engage or disengage (extend or retract).

As seen with syringes 107b and 107c, grooves 129 are configured to end without passing through to a lower surface 134 of port 131 or base member 103. The end point 135 prevents syringes 107b and 107c from rotating sufficiently to be removed from base member 103. In contrast, syringe 107a may be removed while base member 103 and top member 105 are coupled. The grooves 129 associated with the port 131 of syringe 107a extend to lower surface 134. This permits the full unthreading of syringe 107a from base member 103. The other syringes (107b and 107c) can theoretically be removed but would require potentially being inserted prior to the coupling of members 103 and 105.

In operation of assembly 101, it is desired that one or more of syringes 107 are brought into contact or communication with line 117 and attachment device 109 in general. This is done by aligning a syringe with a receiving hole 137 formed in a lower surface 139 of top member 105. Internal seat 127 defines receiving hole 137. When a particular syringe is aligned with receiving hole 137, that syringe may be rotated from a retracted position into an extended position. When fully extended, the top portion of that syringe is seated in internal seat 127. At this time, the syringe is in fluid communication with attachment device 109 through line 117. The plunger 126 of that syringe may be used to discharge fluid into the body of the patient or to withdraw fluid from the body of the patient. As seen in FIG. 1, the syringes may include a saline solution or may be empty awaiting a withdrawal of blood from the patient.

It is important to secure the relative motion of members 103 and 105 when operation of the syringes is happening. To facilitate a restriction on the free rotation of members 103 and 105, assembly 101 includes a protrusion 141 and an indent 143. Protrusion 141 extends outward from lower surface 139. Indent 143 extends inward from top surface 133. Protrusion 141 is configured to seat within indent 143 which in turn stabilizes members 103 and 105 relative to one another sufficient to stop free rotation. Assembly 101 may include one or more protrusions 141 and indents 143. It is preferred that a sufficient quantity of each are present to permit a restraint on free rotation with each syringe that engages with internal seat 127.

The method of exchanging fluids through a CVC hub includes coupling an attachment device to the CVC hub. The attachment member is coupled to the top member at the second end. The plurality of syringes are located within the base member. The base member is in communication with the top member. The top member and the base member are configured to rotate along opposing surfaces. The user rotates the base member relative to the top member to selectively align one of the plurality of syringes with the receiving hole and second end of the attachment device. The syringe is actuated into selective engagement with the second end of the attachment device for the exchange of fluids. The attachment device remains in contact with the CVC hub as the plurality of syringes selective rotate about the top member and alternate in selective engagement with the second end of the attachment device.

The method of using assembly 101 involves the single connection with the CVC hub via the attachment device with the simultaneous sequential engagement of multiple syringes through rotation of the base member relative to the attachment device and top member. Operation of the syringes is used in a predefined order wherein the set of syringes are rotated about the axis and brought in selective communication with the attachment device. Each syringe is rotated to engage with the attachment device and subsequently rotated for disengagement. When all are disengaged, rotation of the base member is possible. At least one syringe is fully removable from the base. Ultimately the invention may take many embodiments. This assembly overcomes the disadvantages inherent in the prior art.

A more detailed example of steps may be shown through the process below:
1) coupling an attachment device to the CVC hub;
2) choosing a syringe from a plurality of syringes held within ports in a base member;
3) rotating the base member relative to a top member to align one of the plurality of syringes with the attachment device;
4) engaging a first syringe of the plurality of syringes with the attachment device to flush the CVC hub with saline;
5) discharging fluid from the first syringe through the attachment device;
6) drawing waste fluid into the first syringe through the attachment device;
7) disengaging the first syringe;
8) rotating the base member so as to align a second syringe with the attachment device;
9) engaging the second syringe with the attachment device and withdrawing blood through the attachment device and into the second syringe;
10) disengaging the second syringe;
11) reengaging the first syringe with the attachment device by rotating the base member to realign the first syringe with the attachment device;
12) discharging the waste fluid through the attachment device; and
13) engaging a third syringe of the plurality of syringes with the attachment device to flush the CVC hub with saline.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

What is claimed is:

1. A method of exchanging fluids through a CVC hub, comprising:
coupling an attachment device to the CVC hub, the attachment device coupled to a top member at a second end;
locating a plurality of syringes within a base member, the base member in communication with the top member, the top member and the base member configured to rotate along opposing surfaces;
rotating the base member relative to the top member to selectively align one of the plurality of syringes with the attachment device; and
actuating the plurality of syringes into selective engagement with the second end of the attachment device for the exchange of fluids;
wherein the attachment device remains in contact with the CVC hub as the plurality of syringes alternate selective engagement with the second end of the attachment device.

2. The method of claim 1, wherein at least one of the plurality of syringes is removable from the base member.

3. The method of claim 1, wherein the plurality of syringes engage with the second end by rotating within the base member.

4. The method of claim 1, wherein the exchange of fluids includes reinjecting waste fluid back through the attachment device from at least one of the plurality of syringes.

5. A method of exchanging fluids through a CVC hub, comprising:
coupling an attachment device to the CVC hub;
choosing a syringe from a plurality of syringes held within ports in a base member;
rotating the base member relative to a top member to align one of the plurality of syringes with the attachment device;
engaging a first syringe of the plurality of syringes with the attachment device to flush the CVC hub with saline;
discharging fluid from the first syringe through the attachment device;
drawing waste fluid into the first syringe through the attachment device;
disengaging the first syringe;
rotating the base member so as to align a second syringe with the attachment device;
engaging the second syringe with the attachment device and withdrawing blood through the attachment device and into the second syringe;
disengaging the second syringe;
reengaging the first syringe with the attachment device by rotating the base member to realign the first syringe with the attachment device;
discharging the waste fluid through the attachment device; and
engaging a third syringe of the plurality of syringes with the attachment device to flush the CVC hub with saline.
